(19) United States
(12) Patent Application Publication
(10) Pub. No.: US 2017/0112664 A1
(43) Pub. Date: Apr. 27, 2017

(54) Eye drop warming apparatus

(71) Applicant: Steven Michael Ferkel, Belleville, MI (US)

(72) Inventor: Steven Michael Ferkel, Belleville, MI (US)

(21) Appl. No.: 15/338,641

(22) Filed: Oct. 31, 2016

Related U.S. Application Data
(60) Provisional application No. 62/249,963, filed on Nov. 3, 2015.

(51) Int. Cl. A61F 9/00; H05B 3/00

(52) U.S. Cl. CPC A61F 9/0008; H05B 3/0014; H05B 2203/021

(57) ABSTRACT

A plug in electronic device which warms and protects liquid ocular treatments stored in disposable containers. This device continually keeps your eye drops warm and ready for ocular administration while also continually keeping the treatments protected by anti bacterial light emitted from a LED. This device is to be used with disposable eye drop dispensers which fit within, upon, or around the surface area of a temperature changing element integral to the inventions structure.

### FIELD OF INVENTION

This invention relates to the medical device field, cosmetic field, home health care field and consumer electronics field.

### BRIEF DESCRIPTION

A compact plug in electronic device that reduces the initial shock associated with administering liquid ocular treatments dispensed from disposable eye drop bottles. This device warms the liquid ocular treatments with use of one or more resistors and also protects the treatments from bacterial contamination with the use of at least one anti bacterial light.

### BACKGROUND OF INVENTION

The idea of putting eye drops into the eyes for administering medication ocularly can be traced back to Fort Worth, Tex. USA, 1950. Conner and Alexander, both pharmacists, joined their names together in 1945 when they formed “Alcon”. Alcon introduced the first eye drops for the general public and also patented the eye drop bottle that we still use today. In 1950 Alcon introduced two different eye drops into the market, one for minor eye infections, and one for red itchy eyes. In 1953 Robert Alexander and a local physician create and patent an eye drop dispensing bottle they named the “DROP-TAINER”. Different types of diseases and disorders are now treated ocularly with the use of eye drops dispensed from such “DROP-TAINER” style dispensers, even those disorders that have nothing to do with the eyes.

The application of a modern day eye drop bottle is simple and millions of people worldwide use them daily. Open the container, tilt your head back, aim the nozzle down toward your eyeballs and squeeze. Many different eye drop dispenser designs have been patented which are for the utility of enhancing or changing the experience of using eye drops. Bottles have been designed with protruding nose and brow rests and some incorporate line up markings for aiming the drops more accurately.

Recently, single dose liquid containers used in treating ocular symptoms have become very popular among consumers. These single dose containers are a onetime use disposable embodiment which has no cap and is opened by the twisting and tearing open of a thermo plastic hermetic seal. These little disposable eye drop dispensers are employed to reduce the risk of cross contamination of the liquid medication stored in the containers. In most cases these single use dispensers have a printed lot or batch number attached to them as well as printed information about the medication, its expiration date and any other important information about its contents that the manufacture feels is necessary.

Using liquid eye drops is a common daily activity for many people. The problem with using eye drops is the initial unpleasant feeling one gets from the drops hitting the eyeball. It's so unpleasant for some people that they apply the drops to the inside corners of the eye to avoid the feeling. Applying eye drops to the corners of the eye causes another problem altogether which many inventors have tried to solve. Aiming and getting the drops to that small corner area of a person's eyes while avoiding direct contact with the eyeball can be very tricky. A smaller target area for the drops complicates the whole process. If one had the option of just eliminating the uncomfortable feeling of the drops being directly placed on the eye ball then aiming the drops becomes less of an issue and the user of the liquid could confidently squeeze the bottle, aiming more loosely without fear of that shocking and discomforting feeling that is associated with administering these types of liquids to the eye.

With eye drop liquid available in every drug store in America, it is obvious that millions of people buy and use these liquids. I can't help but wonder how many more people out there would start using these liquids on a more regular basis if it was a more enjoyable experience. I also have to wonder how many people are avoiding using these liquids altogether because they just can't stand the feeling. Plainly put, what is needed is a solution for eliminating the shock and discomfort associated with using eye drops.

### BRIEF DESCRIPTION OF THE SEVERAL VIEWS OF THE DRAWINGS

FIG. 1A shows elevated isometric view of preferred embodiment of invention plugged into a household electrical outlet.

FIG. 1B shows elevated isometric view of the preferred embodiment of invention separated into its two main parts.

FIG. 1C shows an isometric exploded view of preferred embodiment's liquid container.

FIG. 2A shows a front side view of preferred embodiment of invention.

FIG. 2B shows left side view of preferred embodiment of invention (right side same).

FIG. 2C shows back side view of preferred embodiment of invention.

FIG. 2D shows elevated isometric back side view of preferred embodiment of invention.

FIG. 3A shows an elevated isometric view of preferred embodiments warming part.

FIG. 3B shows a perspective view of the inventions warming part with its front cover removed exposing its internal electrical components.

FIG. 3C shows an isometric perspective view of the preferred embodiments electric warming circuit removed from the inventions shell.

FIG. 3D shows an electrical diagram of preferred embodiment's electrical circuit.

FIG. 4A shows an elevated isometric view of preferred embodiments warming part.

FIG. 4B shows a perspective view of the inventions warming part with its front cover removed exposing its internal electrical components.

FIG. 4C shows an isometric perspective view of the preferred embodiments electric warming circuit removed from the inventions shell.

FIG. 4D shows this embodiments electrical diagram.

FIG. 5A shows a perspective view of the preferred embodiment in union with the liquid dispenser.

FIG. 5B shows an elevated perspective view of the inventions warming part.

FIG. 5C shows a front perspective view of the inventions warming part with the front cover removed and its internal electronics exposed. In this slightly modified form the heating component is mounted along with the two diodes to a circuit board.

FIG. 5D shows a front planar view of the same.

FIG. 5E shows this embodiments electrical diagram.

FIG. 6A shows an isometric view of a modified form of invention with both the warming part and bottle in union.

FIG. 6B shows this modified form and reveals that the embodiment's two main parts are separable.

FIG. 6C shows an exploded view of this modified form.

FIG. 6D shows a top planar view of this modified forms circuit board.

FIG. 6E shows this modified form's electrical diagram.

FIG. 7A shows a frontal view of inventions bottle forming mold with its depression forming part in a particular phase of action.

FIG. 7B shows a frontal view of inventions bottle forming mold with its depression forming part in a particular phase of action.

FIG. 7C shows a frontal view of inventions bottle forming mold with its depression forming part in another particular phase of action.

FIG. 7D shows an elevated perspective view of Inventions depression forming part.

FIG. 8A shows an elevated front perspective view of one stage of inventions bottle being formed in a blow molding mold.

FIG. 8B shows an elevated front perspective view of another stage of inventions bottle being formed in a blow molding mold.

FIG. 8C shows yet another perspective view of another stage of inventions bottle being formed in a blow molding mold.

FIG. 9A shows a modified form of inventions container part from a raised ¾ perspective view in a semi disassembled state. Shown is the liquid containing part of invention and beneath it a clear plastic sleeve having an integral heating element attached to it ready for permanent insertion into the containers concave depression.

FIG. 9B shows a ¾ raised perspective view of modified form of inventions liquid containing part showing a newly affixed heating element.

FIG. 9C shows a ¾ raised perspective view of modified of invention with the newly reconfigured dispenser resting over and in union with its docking station part.

FIG. 10A shows a front raised perspective view of a modified form of inventions warming part. Shown are multiple disposable liquid containers above this modified form ready for insertion into inventions warming area.

FIG. 10B shows a ¾ perspective view of this modified form having its front panel removed as well as its lid. Its back panel that protects the electric components is removed revealing the warming parts electrical components and how they are wired together.

FIG. 10C shows a raised ¾ perspective view of modified form of warming part. The warming parts closable lid is closed and multiple single dose containers are shown stored in the warming area of the device.

FIG. 10D shows a top planar view of this modified form closed and containing multiple single dose containers.

FIG. 10E shows a planar left side view of the same.

FIG. 10F shows a planar right side view of the same.

FIG. 11 shows a flowchart for yet another modified form of this invention which incorporates more advanced electronic components with more user options and functions.

FIG. 12A shows a perspective view of a modified form of the invention in its closed and warming state.

FIG. 12B shows a perspective view of this modified form open and ready for its cradled dispenser to be removed by the user.

FIG. 12C shows a perspective view of the invention with its container lifted out of its rotatable container cradle.

FIG. 12D shows a rear perspective view of this modified form's dispensing container. A rear half elliptical window with attached RFID chip is shown in the containers cap. The window is incorporated into the containers cap to allow germ protection LED light to shine into the containers drip nozzle area while the container is cradled inside the device.

FIG. 12E shows a front perspective view of the same container.

FIG. 12F shows this modified form's container from a rear perspective view with its snap on cap lifted above the liquid container.

FIG. 12G shows a left side view of this modified form in open position with its container in its cradle.

FIG. 13A shows an exploded front perspective view of this modified form of invention.

FIG. 13B shows an exploded rear perspective view of this modified form of invention.

FIG. 13C shows a front perspective view of this modified form assembled and closed.

FIG. 14A shows a front perspective view of inventions front console, the rotatable cradle has been removed to show the position of this embodiment's temperature sensor, plunger button and chip reading antenna.

FIG. 14B shows a rear perspective view of inventions circuit board and electrical components.

### DETAILED DESCRIPTION

Using liquid eye treatments that are administered ocularly is both a physical and mental experience for the user. My invention creatively solves two problems associated with administering such liquid treatments ocularly. The first thing my invention does is solve the problem of the initial shock and discomfort associated with using liquid treatment administered ocularly. The second thing my invention does is protect those liquids and their containers from unwanted bacterial growth with the use of radiation emitted from a LED light source.

This new invention has to do with comfort, safety, convenience, and giving the consumer options for their eye care that, before this invention, did not exist. Prior to my invention, there was no specific device available for eliminating the unwanted muscle reflex discomfort associated with receiving eye drops directly to a person's ocular area. Prior to my invention, people using eye drop liquid would have to apply the liquid at room temperature whatever that temperature may be. The lower in temperature the eye drop liquid is from 98 degrees Fahrenheit the greater initial shock to the eyeball due to the fact that most people's core body temperature is 98.7 degrees and the shock occurs due to the difference in the contained liquids temperature and the body's core temperature. If the room temperature happens to be 98 degrees or warmer the eye drops will produce little to zero initial shock when applied if the eye drop liquid has had time to acclimate to room temperature but that's no help because most people do not keep their room temperature at 98.7 degrees. It is my opinion that there are a large number of people that would prefer eye drops pre warmed and readily available to them in that warmed state because it actually makes using the eye drops a pleasing experience. The shock from using these types of liquids at room temperature can cause unwanted muscle reflex of the neck, shoulder, and back muscles which can cause increased distress and discomfort to those suffering from neck and back injuries. With my invention, those people who prefer a more gentle and pleasing experience while using these types of liquid treatments now have that option.

Because my invention is primarily designed to be plugged into a bathroom power outlet and not all bathrooms are the most sanitary environments, I decided to add a UV producing LED light to help prevent bacteria from growing within and upon my inventions liquid containing part.

I decided to do some research to see if there was an LED light that could kill bacteria. What I found was that there are certain wavelengths of blue light that seem to demonstrate anti bacterial properties. I found that there were some studies done on this subject using 470 nm and although it doesn't destroy bacteria, it is believed by some to have the ability of slowing or stopping the further growth of bacteria that is already present on a surface area. I also found that UVB was found to actually kill bacteria as where the 470 nm range only seems to stop the bacteria from populating. So this invention can incorporate either one of these LED's with the UVB the more preferred LED for those types of ocular treatment which contain substances which will not be adversely affected by UV or UVB radiation with the less destructive 470 nm type being utilized when treatments that contain beneficial bacteria are being heated by this invention. The reason behind this is that certain liquid eye treatments contain actual beneficial biological material which could possibly be rendered ineffective by the UV radiation, and with such treatments, the 470 nm's radiation will provide the perfect alternative as a much less destructive form of protection. knowing that 470 nm can at least stop bacteria from reproducing, I consider the 470 nm the safe alternative to the UV/UVB radiation if and when there is any question as to whether or not the treatment contains beneficial micro organisms or a possible chemical which cannot withstand the UV/UVB radiation.

I believe this invention is ideal for both home use as well as hotel stays. This invention can turn the normally unpleasant routine of using eye drops into an actual pleasant and comfortable experience especially for those with back and neck injury because it eliminates the flinch factor associated with receiving eye drops to the eyeball. Not only will this invention remove the shock associated with using these types of liquid medications, it will also give the user added peace of mind knowing that the liquid has been protected by an anti bacterial light.

This invention is also ideal for hospice care as well as clinical care environments due to its gentle warm soothing nature. This invention will also work well for veterinarian care because animals, like humans, can be extremely sensitive to liquid drops being dropped into their eyes.

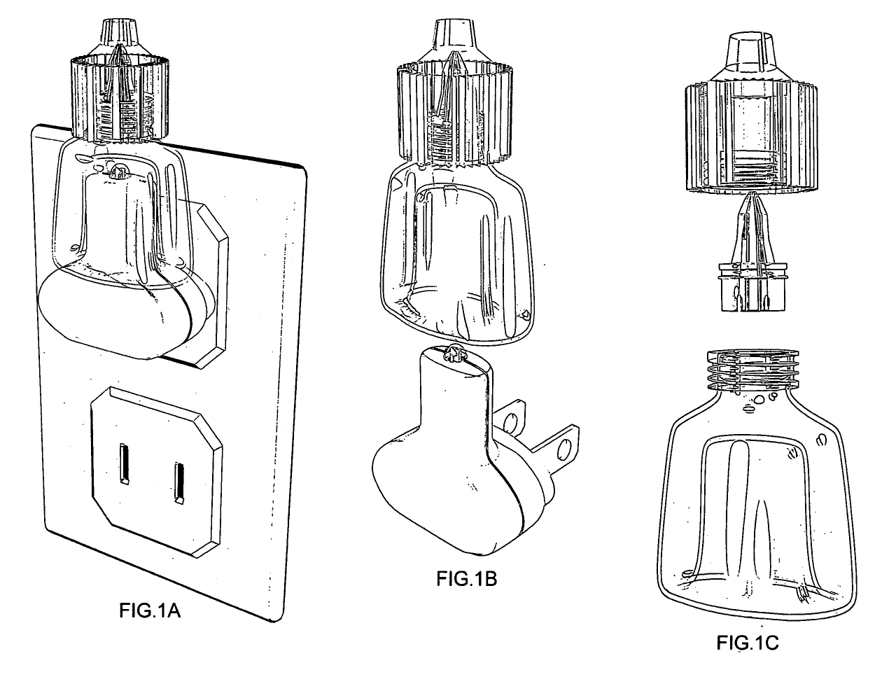

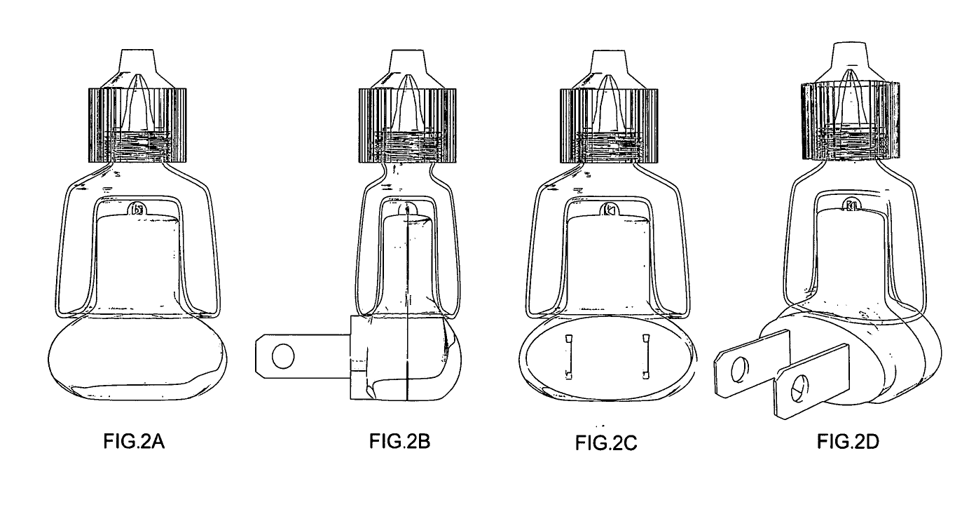

The preferred embodiment of this invention comprises a plug in heating element and a plastic molded bottle shaped so that the bottle rest upon and around the warm able area of the plug in heating element as shown and described in FIG. 1A and FIG. 2A of the several drawings.

The bottle and its contents are warmed by the bottles walls resting for a period of time against the architecture of the plug in heater while the device is plugged in to a power outlet. This warming device comprises at least one heat producing resistor for the warming of the bottle and its contents, a non light emitting diode for rectifying, and a light emitting diode for emitting light upon the surface areas of a treatments container. This invention can be configured for all power sources. Different diodes, diode values, resistors, and resistor values, depending on the peak input voltage you're working with, can be used to oppose the current and produce different watts of heat production from the device.

FIG. 1A shows an isometric view of the preferred embodiment of this invention plugged into a household 110v power outlet. The liquid container is resting upon and around the plug in warming part of the invention. FIG. 1B shows how the container is separable from the devices plug in warming part. FIG. 1C shows the liquid dispensing container in an exploded perspective view. This is to illustrate that embodiment's container is not a single part construction. It is a squeezable liquid containing bottle with a snap and seal nozzle protected by a screw on protective cap. FIG. 2A shows a front side view, FIG. 2B a left side view, FIG. 2C a back side view, and FIG. 2D an elevated rear perspective view of the preferred embodiment.

The liquid dispensing container shown in FIG. 1A and FIG. 2A of the several drawings is shaped most specifically to Increase the compactness of the overall arrangement, increase the efficiency of the warming unit, accommodate the union of the warming part with said liquid dispensing container, stabilize and hold said container in place while it is being warmed by inventions warming part and provide a closer proximity of inventions LED to the dispensers' nozzle improving the concentration of anti bacterial protection in the periphery of said nozzle.

This inventions preferred embodiment's heating part is to be used in combination with a transparent plastic squeezable liquid container. In this embodiment the container is specifically shaped with a concave depression in its central area for accommodating its union with an elongated and protruding electric heating element which rests partially and temporarily within this elongated concave depression when it is warming the liquid. Instead of heating the liquid by the outermost walls of the container receiving heat from a single non surrounding heat source, this embodiment provides a more compact way of the container receiving heat from an electrical circuit. As is shown in FIG. 1A and FIG. 2A of the several drawings, the central area of the bottles containment area has been arranged to allow the insertion of the electric warming part. By having the container rest around the warming unit, less heat escapes the surface area of the warming part. Due to this configuration the heat produced by the resistor has less of an escape route and therefore it is forced to travel through the liquid from the central area of the bottle containment area to the containers outermost walls. Simply put, instead of the heat energy being absorbed and lost by the bottles surrounding air it is now absorbed more directly by the bottle and its contents.

FIG. 1A shows the placement of the preferred embodiment's LED. This is the preferred placement of the inventions LED because of the close proximity of the LED to the containers nozzle which is the only entry point where bacteria could enter the container. With the LED placed in such close proximity to the nozzle, effective amounts of radiation can reach the most desirable area of the container which is its drip nozzle and cap area.

FIG. 2A shows a front side view of the preferred embodiment of this invention with its dispenser attached, FIG. 2B a left side view of the same, FIG. 2C a rear view and FIG. 2D an elevated isometric view of the back side of inventions warming part coupled with the inventions eye drop container.

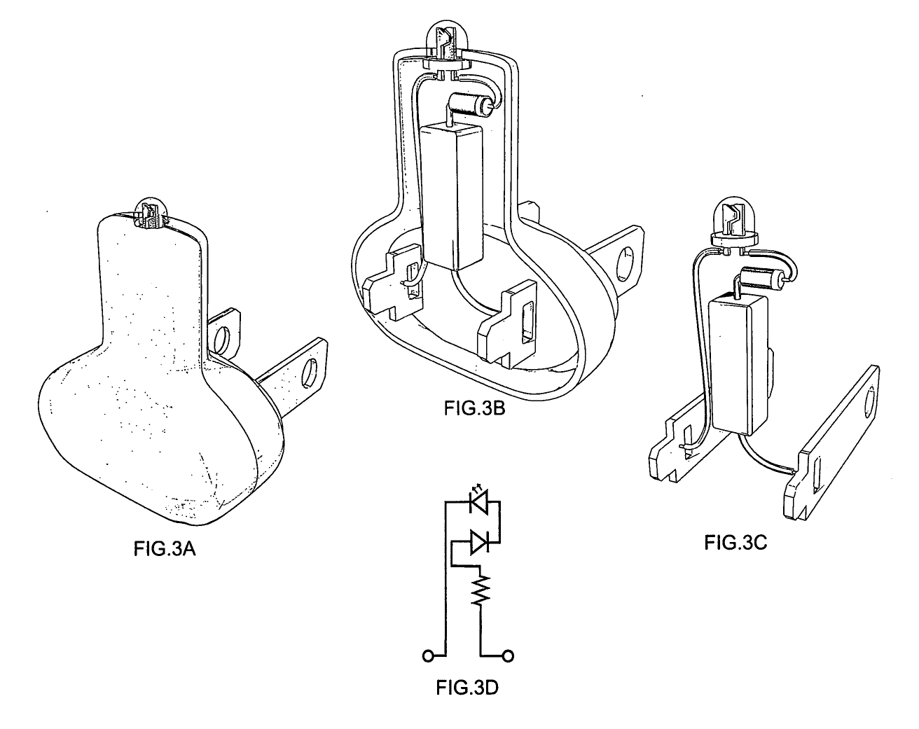

FIG. 3A shows the inventions warming part with its two part shell sealed closed and encapsulating the devices electrical components. FIG. 3B shows an isometric perspective view of the warming part with the front part of its shell removed. The internal components of the warming device are exposed showing that the warmer produces heat with the use of a ceramic coated resistor which is wired to both diodes as well as the two metal male plug prongs. The two power outlet prongs of the inventions warming part extend outward from the inside of the devices shell to allow their insertion into a power outlet automatically turning the device on as it is plugged in.

FIG. 3C shows the circuit without its shelled enclosure. This drawing is meant to show more clearly how it is wired together.

FIG. 3D shows this embodiments corresponding electrical diagram. This diagram does not specify the values of the components because they can vary dependant on the input voltage being used. If in the United States and the device was to be plugged into a 110v-120v power outlet the values could be as such:

The resistor used could be a 6k ohm 5 watt high power ceramic resistor.

The non light emitting rectifier diode would be an IN4003.

The LED would be a blue 470 nm 20 ma or 20 ma UV/UVB LED. (treatment dependent)

These components at these specified values will give you a working circuit that can handle a 110V power source with 30 amps max surge load.

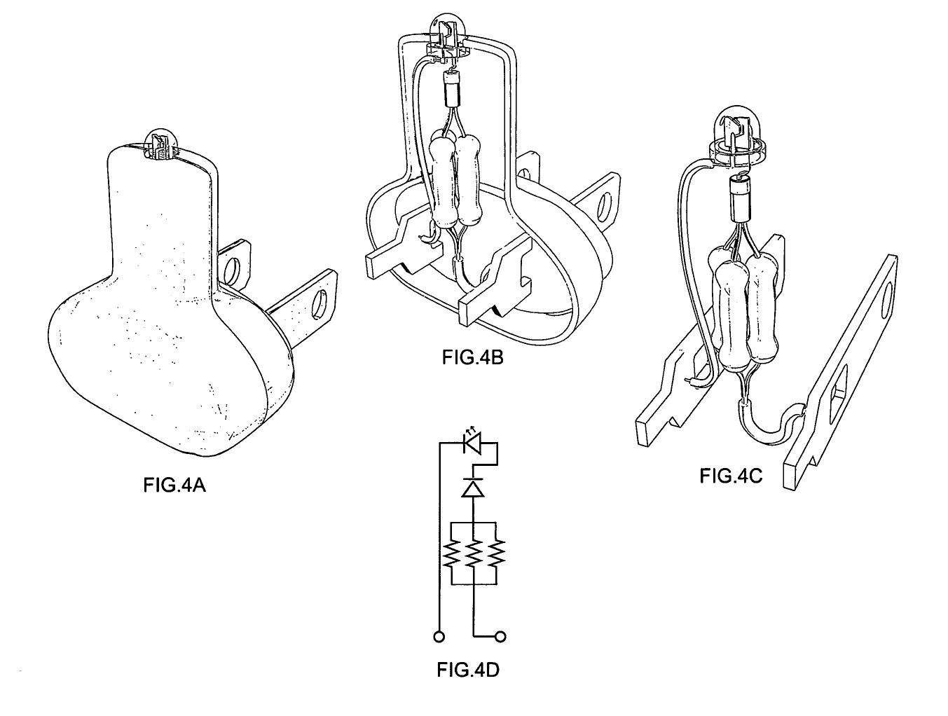

FIG. 4A shows an elevated perspective view of the inventions warming part sealed and encapsulating its internal parts.

FIG. 4B shows an elevated isometric perspective view of the inventions preferred embodiment using three separate resistors rather than one high power ceramic resistor. The front cover of the shelled enclosure is removed to show the wiring of this particular embodiment's resistors, diode, and LED. This drawing shows three 22k ohm ½ watt resistors wired in parallel to an IN4003 rectifier diode, a 20 ma LED and two electric power plug prongs. All three 22k resistors shown are carbon film resistors. FIG. 4D shows the corresponding electrical diagram. The values are not listed on this electrical diagram due to the fact that they can vary dependent upon the peak input voltage of the power source the device is plugged into. The values previously listed in this paragraph will work for a standard household 110v power outlet. This diagram is drawn to illustrate how different kinds of resistors as well as different amounts of resistors can be used to produce heat for the device. FIG. 4C shows the electrical circuit with all of its electrical components removed completely from its shelled enclosure. This figure is shown to simplify and aid in the understanding of how this circuit is wired together.

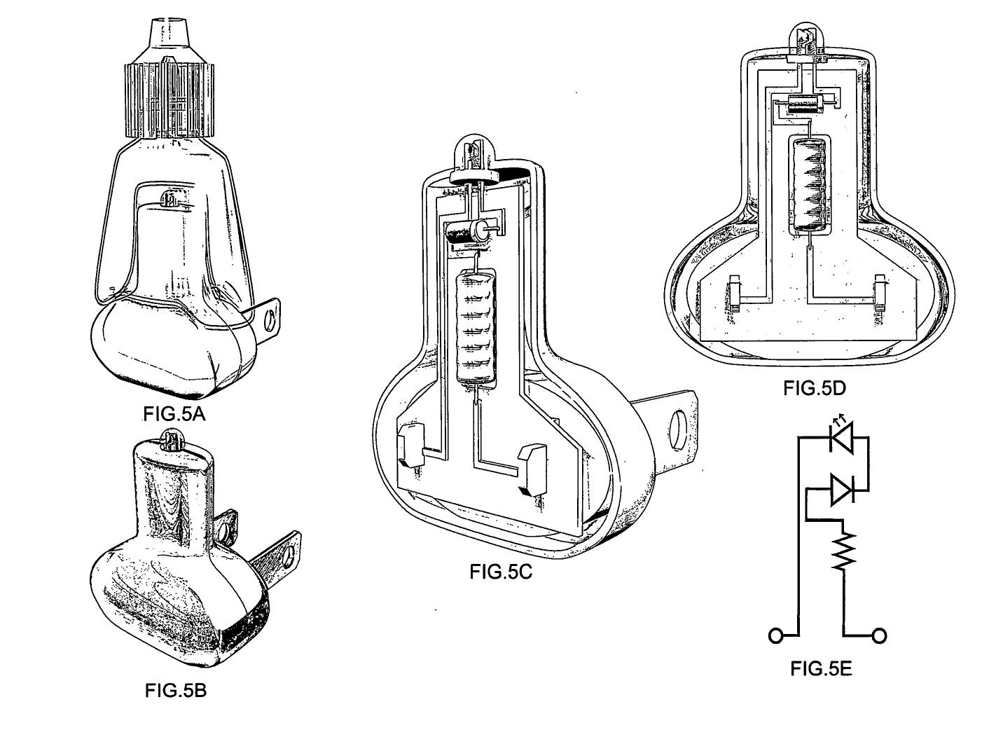

FIG. 5A shows an isometric perspective view of the invention. It shows the complete invention with its bottle and warming part in union. The bottle is of a slightly different shape than the embodiment shown in FIG. 1A but the overall utility remains unchanged. FIG. 5b shows this forms warming part after the bottle has been removed.

FIG. 5C shows an isometric perspective view of inventions warming part with the front part of its shell removed. This embodiment shows how a circuit board can be used instead of just wiring the components together. This view shows a circuit board with a single resistor centrally placed within a rectangle hole in the circuit board which aids in a more even distribution of heat inside the devices shell where the bottle rests in union with this warming part. FIG. 5D shows a front perspective view of this modified form showing the circuit boards conductive leads and the arrangements of the circuit boards components. FIG. 5E shows this embodiments corresponding electrical diagram.

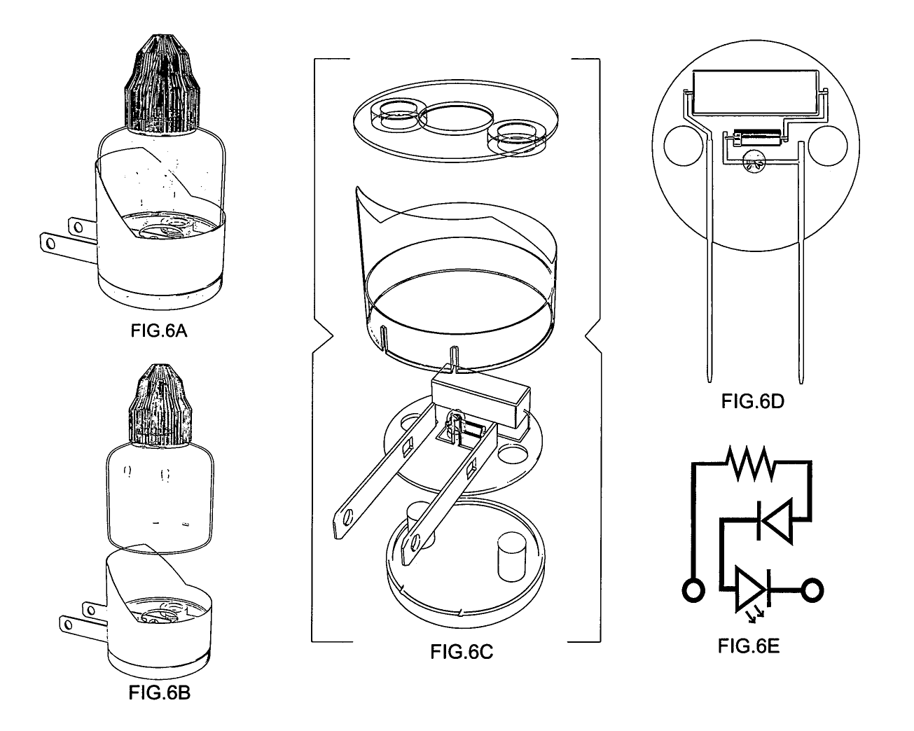

FIG. 6A shows yet another embodiment of this invention. This embodiment does not require a specially shaped container for warming by the inventions circuit but rather uses a simple cylindrical shaped liquid container which rest upon and partially within a walled and shelled enclosure with a planar base. In this embodiment, the heat source comes from a heating circuit which lies beneath the liquid dispenser. With this embodiment, the LED light and heating circuit lies beneath a windowed planar wall integral to the devices shell. This clear plastic window acts as the containers supportable base while also allowing the LED light to shine up thru it and into the area of the liquid dispenser when it is resting upon this planar platform. FIG. 6B shows that the container and warming part are separable. FIG. 6C shows an elevated exploded perspective view of this modified forms warming part. This view shows the circular window transparent to reveal the windows underside and how it fits over the two cylindrical protrusions extending upward from the shells lowest most part. FIG. 6D shows this embodiments circuit board from a planar top view with FIG. 5E showing this embodiments corresponding electric diagram. This embodiment uses a high power ceramic coated resistor of 5 watts 6k ohm, an IN4003 diode and a 20 ma Light emitting diode all of which are soldered to a circular shaped circuit board with two insert able metal power outlet prongs also soldered and integral to this embodiment's circuit board.

When using a single high power ceramic resistor in the preferred embodiment as well as any modified form, you may benefit from the integration of a small metal heat sink for the dissipation of excess heat produced by inventions warming part. If even lower temperatures are desired a lower wattage resistor with the correct ohm rating for the input voltage can be used.

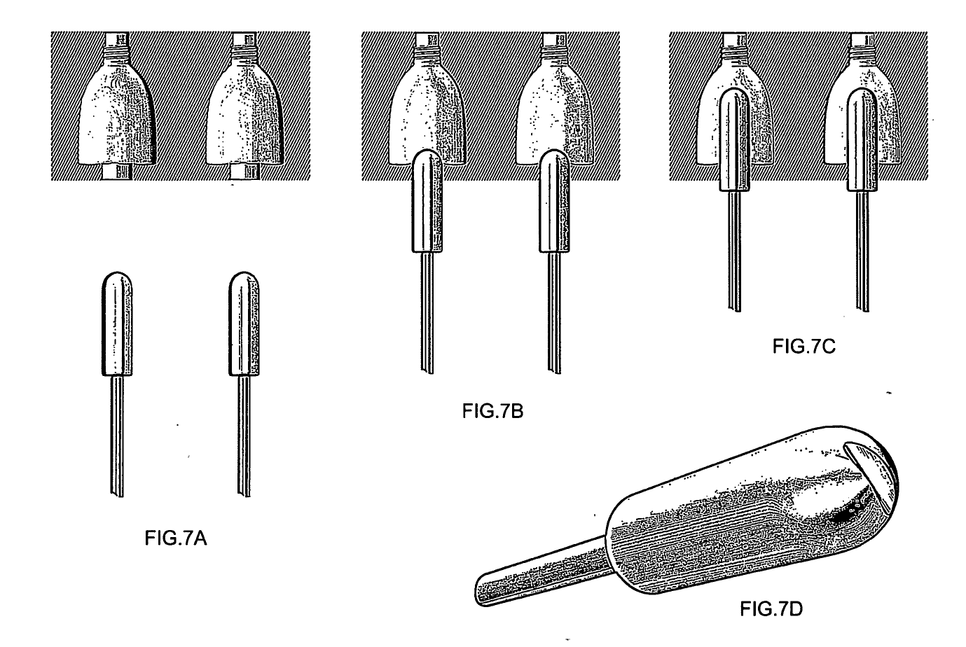

FIG. 7A-FIG. 7D illustrates a part which is used in the manufacture of the inventions preferred embodiments bottle. This part is an elongated depression forming rod which is essential in the forming of the bottles central insertion cavity during the blow molding process. This part is specifically designed to both aid in the forming of the containers elongated heating element containment area as well as aid in the bottles transportation during the manufacturing process. This depression forming rod is lower able and raise able thru ought the mold cavities container forming area. During the blow molding process a parasol is extruded, crimped together at one end, cut and then allowed to drop into the mold cavity. FIG. 7A shows a double bottle mold with two of these depression forming rods in their lowered position. FIG. 7B shows the two rods raised to the position where the cut and crimped parasol is ready to drop into the mold cavities. FIG. 7C shows the depression forming rods raised to their highest position. This position is reached as the parasol is blown into the shape of the mold. As the top part of the mold receives compressed air for the blowing of the parasol into the shape of the bottle, the depression forming rod raises from the position shown in FIG. 7B to its position shown in FIG. 7C forming the depression as the bottle is being formed. FIG. 7D shows an elevated perspective view of the inventions depression forming rod. FIG. 7D is meant to show its divot in the depression forming rod which is designed to catch and hold steady the crimped end of the cut parasol as it drops into the mold cavity preventing the parasol from slipping to one side or the other inside the mold cavity when being dropped into the mold.

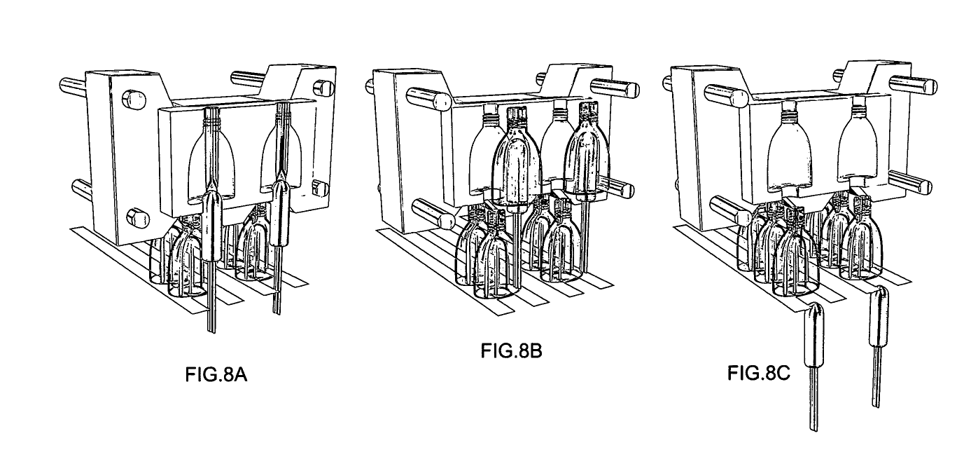

FIG. 8A-FIG. 8C show three elevated perspective views of a double bottle mold assembly. Below the double mold there are two elongated assembly line rails which the bottles are lowered to by the depression forming rods after the bottles are blown. In all of these figure drawings the front platen and mold assembly has been excluded so it doesn't block the view.

FIG. 8A shows two mold cavities with two parasols crimped, cut, and inserted. FIG. 8B shows that the bottles have been blown and the depression forming rods have been raised to their highest position forming the bottles elongated depressions. The bottles have been blown to their full form and the platen has been moved back leaving the bottles positioned upon the two depression forming rods centrally between both halves of the mold platens. FIG. 8C shows that the formed bottles have been removed from the depression forming rods upon contact with the assembly rails beneath the platen and mold assembly. Both rods have been lowered beneath the assembly rails, the bottles being caught by said rails as the rods slip thru the central area of the rails. After the bottles are positioned onto the transportation rails by the depression forming rods they are moved down the line for trimming of the bottles excess plastic. After the bottles are moved down the line the depression forming rods raise back up, the mold is closed, new crimped and cut parasols are dropped into the mold cavity and the blow molding process starts over.

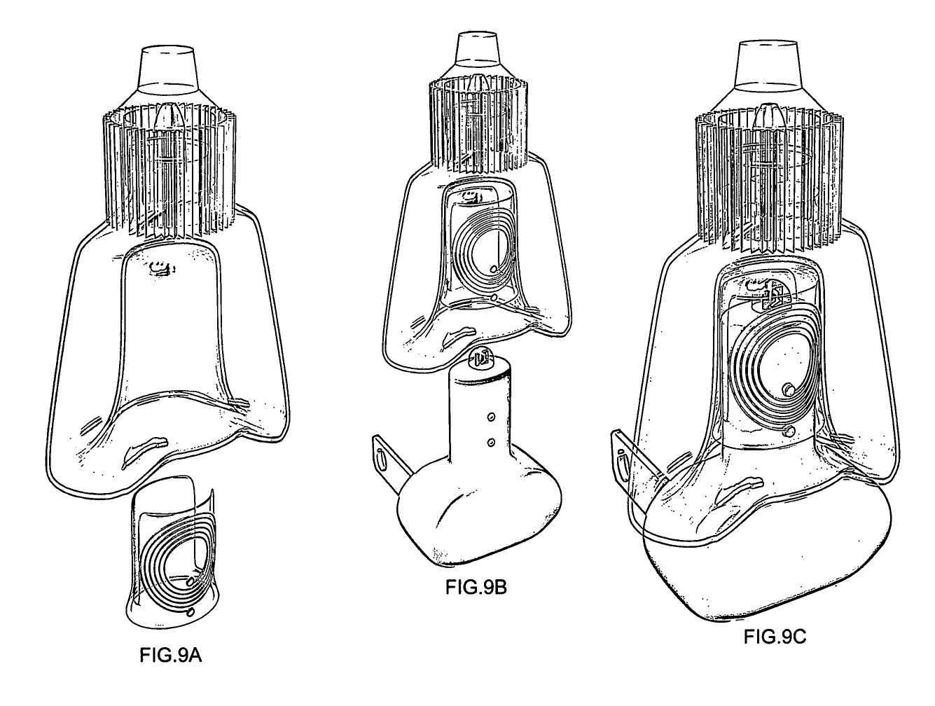

FIG. 9A-FIG. 9C shows a modified form of this inventions preferred embodiment slightly rearranged. Instead of the heat producing element remaining fixed within the shelled plug in part of this invention, it is attached instead to the container, making both the bottle and the heating element one single disposable part. With this modified form, the plug in warmer becomes a docking station and power bridge. This docking station transforms and re routes the power from a power outlet before it is sent to the heating element by the bridging of its circuit while it is docked and in union with the plugged in part.

FIG. 9A shows a coiled heating element attached to a clear plastic sleeve before it is inserted into the concave depression of the preferred embodiment's liquid containing part where it is attached permanently to the liquid container part of the invention. FIG. 9B shows that the heating element part and the container part have been joined and are ready to come in physical contact with what can now, in this embodiment, be referred to as a docking station. The docking stations job is to reduce and reroute the current coming in from the power outlet before it is sent to the heating element. The voltage and current are reduced and then sent to the heating element via two metal connection points where the docking station comes in contact with the architecture of the liquid containers warming element. When in union, a total of 4 connection points between the two parts are used for bridging the circuit. (Two on the container, two on the docking station) These types of connection points can be configured in many ways with recessed metal connections being an option as well as various male/female type plugs also being an option for bridging the circuit between the docking station part and the containers heating element part. FIG. 9C shows the dispenser in union with what is now the docking station part of this modified embodiment. The docking station supports the same preferred placement of the LED light as the warming part of the preferred embodiment does. The main difference between this modified form and the preferred embodiment is the placement and arrangement of the inventions heat producing element. In this modified form the resistor is now a permanently attached part of the container instead of being integrated into the inventions plug in part. With this modified form an additional resistor can be used for stepping down the voltage before it is bridged to the heating element by way of its power leads making contact with each other. The implementation of an additional resistor will allow the heating element of the container to heat up using a much lower voltage while also protecting the operator or handler of the device from dangerous electric shock if they handle the docking station with wet hands.

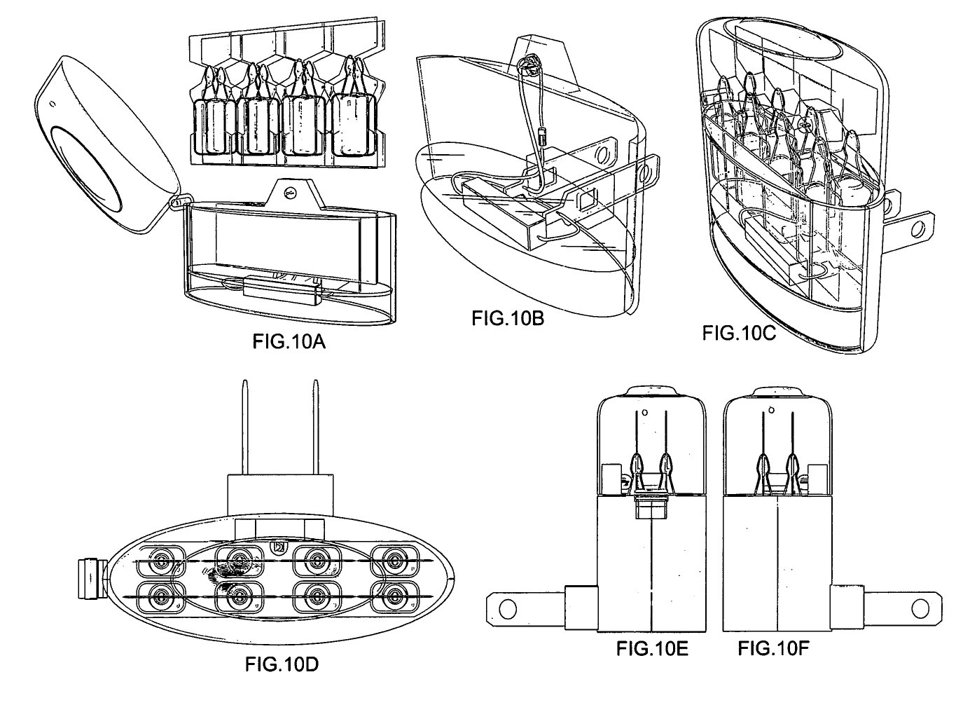

FIG. 10A-10F shows yet another modified form of this invention. This embodiment incorporates a larger warming area for the warming of more than one liquid container at a time. FIG. 10A shows a front raised perspective view of this embodiments warming part. Shown are multiple disposable liquid containers above this modified form ready for insertion into the devices warming area. FIG. 10B shows a ¾ perspective view of this modified form having its front panel removed as well as its lid. This embodiment's back panel, which covers and protects the devices electric components, is removed revealing the warming parts electrical components and how they are wired together. FIG. 10C shows a raised ¾ perspective view of modified form of warming part. The warming parts closable lid is closed and multiple single dose containers are shown stored in the warming part. FIG. 10D shows a top planar view of this embodiment closed and containing multiple single dose containers. FIG. 10E shows a planar left side view of this embodiment containing multiple disposable treatment dispensers with FIG. 10F showing a right side planar view of the same. With this embodiment, a closable capsule with a base stores and warms multiple disposable ocular treatment containers while keeping them sterile with the use of a UV/UVB LED. This embodiment has the addition of a formed enclosure encapsulating the containers being warmed and offers even further protection of the liquid containers from dust and other possible contaminants. This enclosure further provides an additional thermal barrier for improved retention of the heat produced by the inventions high power ceramic resistor which resides in this enclosure separated from the liquid containers by a thin plastic divider which also acts as a support base for the liquid dispensers while they reside in the enclosure. Heat from a high power ceramic resistor is transferred thru and beyond this thin platform into the enclosed warming area of the liquid dispensers. If necessary, vent holes and directional ducts can be incorporated into this platform for better distribution of the heat coming from the resistor.

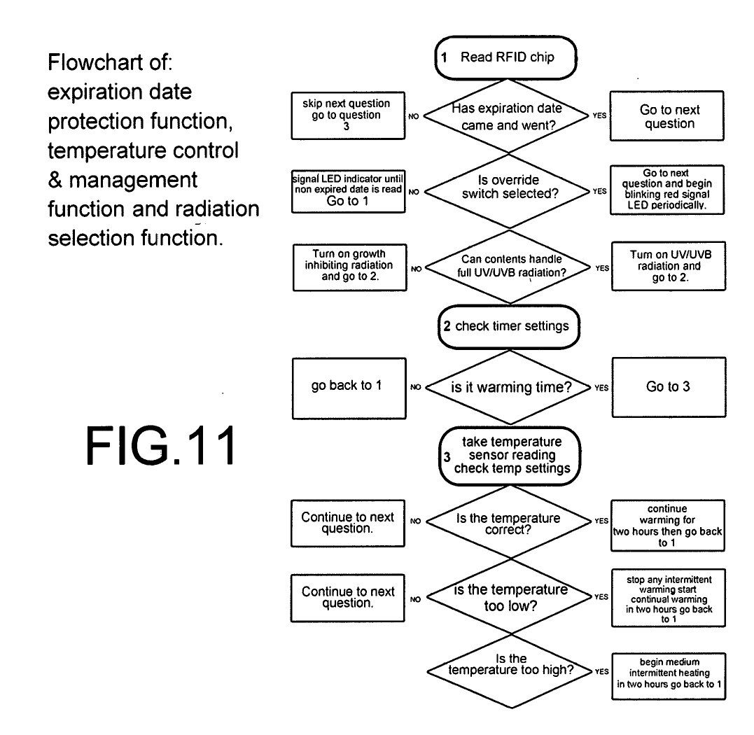

FIG. 11 shows a flowchart for use with a much more involved modified form of this invention. This modified form contains a microcontroller which processes collected data from a temperature sensor, an RFID receiver, a plunger button, an override switch, as well as two separate programmable selection banks (one for timing functions, one for temperature preferences). All of these components work in conjunction with the devices microcontroller and heating element. A display screen has been added for displaying the time of day, the date, and the devices current mode. This display can also display other information such as dosage rate and type of ocular treatment. This embodiment has two different LED's (one for stopping bacteria/one for killing bacteria). This will allow selective means between an LED which kills bacteria versus an LED which merely stops any bacteria from procreating. Three separate temperature settings are shown and are selectable by the user pushing the buttons; one being LOW, one being MEDIUM, and one being HIGH. Whatever selection the user makes, the device will monitor and make adjustments to match. This embodiment also has additional settings for programming the time of day that the device is warming the container for the sake of energy conservation. After the time of day selection has been made the device knows when it's time to start warming. After the device has established its time to warm it does a temperature check and then compares it against the stored temperature selection. After the device has established that the drops are not expired and that the proper radiation selection has been made, it takes a temperature reading and then one of three stored algorithms is chosen to run based on the answering of one of these three questions; Is the temperature correct for what's been selected? Is the temperature colder than what's been selected? Is the temperature hotter than what's been selected? Based on the answering of these three questions the program knows which algorithm to run to get the desired result which is; liquid warmed to the state that the user initially desired and selected. The use of three pre-programmed algorithms, two algorithms making the heater warm at two different intermittent speeds while the other algorithm makes the heater run continuously, are used for keeping the temperature in check. All three of these algorithms are used to adjust the temperature by the program re-running once every 2 hours making adjustments by choosing one of these three algorithms every two hours based on the readings of the temperature sensor and the cross referencing of that reading with the previous selections made by the user. The RFID components of this embodiment provide the means of acquiring specific information from different types of liquid treatments. In this embodiment the container has been equipped with a passive RFID chip for communicating its product information to the stationary part of the invention. This embodiment uses the date information it gathers from the RFID chip to signal to the user whether or not the contents have expired. In this embodiment the device will not heat the container if the expiration date has passed without the further selection of an override button. If the date has indeed passed and the override button has been pushed, the device than uses the information off the chip to determine which radiating means to use. The devices RFID receiver reads the product information and then sends it to the processor which then determines, based on the information it reads, whether or not the contents can handle strong UV/UVB radiation without being adversely affected. If the liquid contents cannot handle the UV radiation than the device chooses and uses the other less damaging radiation means and vice versa.

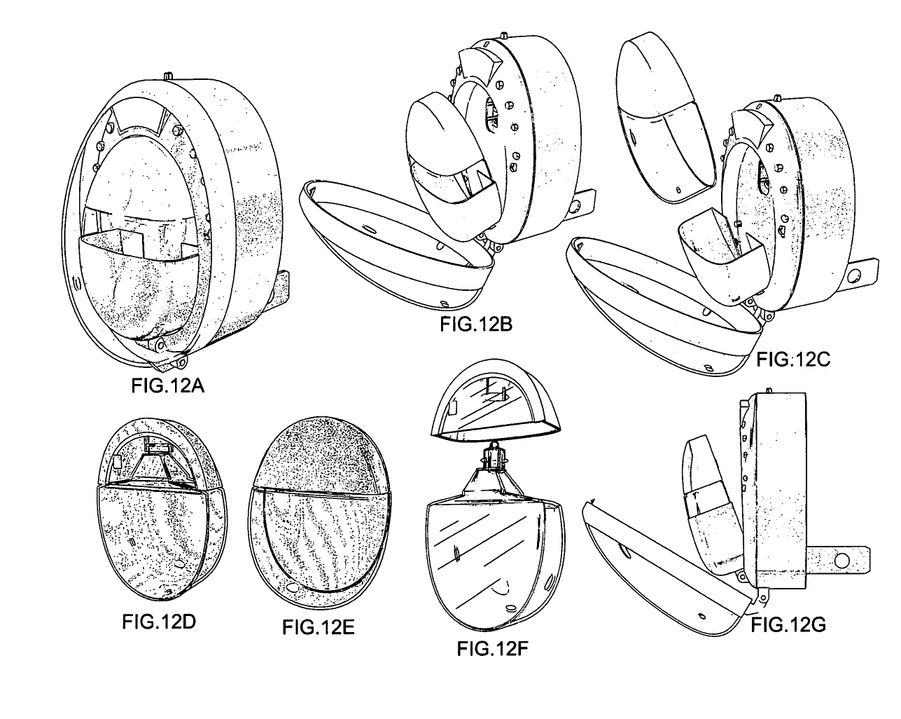

FIG. 12A-12G shows several different perspective views of this more involved embodiment. FIG. 12A shows a perspective view of the invention in its closed and warming state. The liquid treatment is contained within a removable squeezable eye drop dispenser which itself is resting in a pivot able cradle which is pushed back and sealed within the warming area of the device by the closing and latching of the devices front domed and hinged cover. FIG. 12B shows a perspective view of this embodiment opened and ready for the dropper bottle to be removed. The small button on top of the device has been pressed, releasing the latch. With the help of gravity, the front domed cover has rotated forward opening the device. As the front domed cover rotates down it frees the cradle to rotate down and at an angle which makes the dispenser easier to remove from the device.

FIG. 12C shows the dispenser lifted away from the inventions cradle. FIG. 12D shows a rear perspective of this modified forms liquid container. FIG. 12E shows a front perspective view of this embodiment's liquid container. The rear side view shown as FIG. 12D shows a half elliptical transparent window in the container's cap with a square passive RFID microchip attached to it. FIG. 12E shows a front perspective view of same container and FIG. 12F shows another perspective view of this embodiment's container with its cap removed showing that it is indeed a dropper style dispenser with a removable cap. FIG. 12F shows a left side view of this embodiment with its front dome cover open and the container's cradle rotated forward allowing the container to be available to the user at a good ergonomic angle. This view is also a good representation of the double hinge design of this particular embodiment.

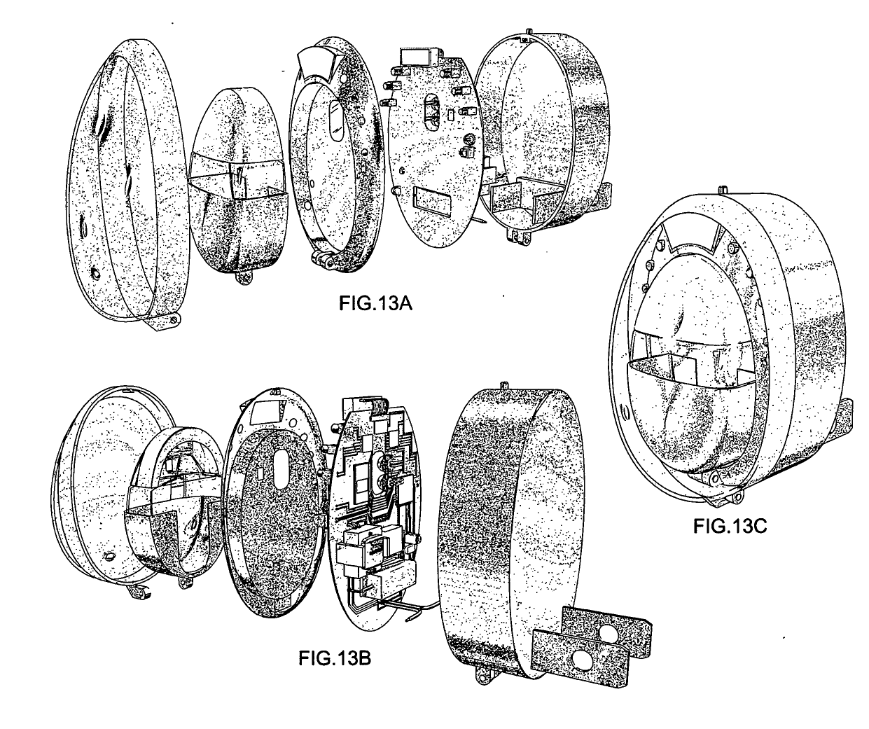

FIG. 13A shows a front perspective view of this embodiment exploded into its five main parts. The shelled enclosure comprises; (from left to right), a domed and hinged clear front cover, a rotatable dispenser cradle, a console panel having a depression shaped to receive and partially surround both the cradle and the treatment dispenser, a circuit board comprising all of the electronic components, and lastly, the rear cover which has power plug prongs integral. FIG. 13B shows the same exploded embodiment from a rear perspective view. FIG. 13C shows a front perspective view of the device assembled, closed and it its warming state.

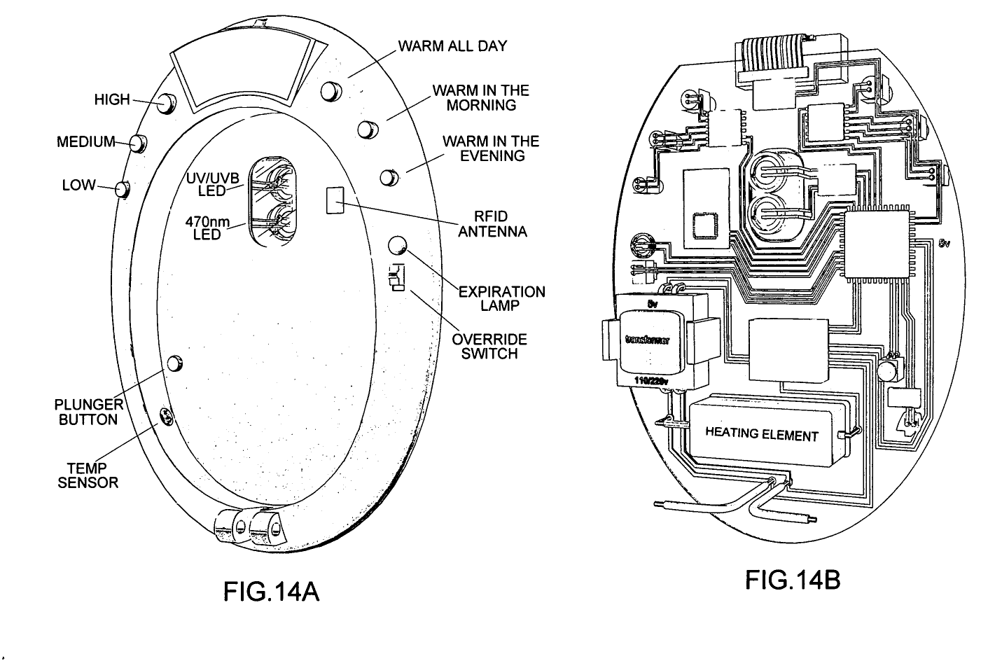

FIG. 14A shows the front console panel of this modified form. The position of the temperature sensor is shown as well as the placement of the RFID reader's antenna and the plunger button which keeps track of how many times the device is opened. The buttons are labeled how they might be arranged and the override switch with the red LED indicator lamp position is also shown. FIG. 14B shows the placement of the RFID chip reader; the microprocessor, the selection banks, as well as other electrical components. FIG. 14A and FIG. 14B are to be interpreted simply as examples of the arrangement of various components and should not be construed as carved in stone because there are many different possible arrangements of the electrical components which can achieve the same results. This arrangement is only to give a general idea of how it might be arranged. Different types of transformers like a buck converter might be used; A transformer less power supply could even be used as well as different types and amounts of heat producing resistors for warming the treatments. FIG. 14A and FIG. 14B are drawn primarily to show the desired physical position of the chip reader, the button selection banks, the inventions two LED's, the temperature sensor, the main heating source and the plunger button which keeps track of the opening and closing of the device.

Every form of the invention shown and described in this specification, from the preferred embodiment to the last of the modified forms, are within the spirit and scope of this invention which is a device specifically designed for the utility of relieving the discomfort and shock that comes with using eye drops at room temperature with the further utility of keeping those liquids safe from contamination while they are being stored and warmed in their containers. Every embodiment shown incorporates electric heating means and they are all designed to withstand being plugged into a power outlet 24/7 keeping these liquids continually warm and ready to administer at any time and without any extra effort or wasting of water by the user. Precious resources such as time, water and electricity will be conserved compared to any alternative ways one currently has available to them for the warming of these types of small containers. My invention takes no time because it's always plugged in keeping the liquids warm and ready to administer at a moment's notice. This invention opens up new possibilities for many people who have to take their eye drops but absolutely hate the feeling of using these types of liquid treatments.

This inventions plug in shelled parts and enclosures can be made from plastic, glass, carbon fiber, metal, rubber or a combination of these materials. All forms shown using a circuit board are for those who prefer using PCB type circuits although it will be dependent upon the embodiment and whether or not it's going to be mass produced. For mass production and for automation and assembly purposes the use of a circuit board is always an option. If making this invention with only warming capability you only need to use a single resistor. No rectifier diode or LED is needed to make this invention provide its discomfort relieving function. For mass production an injection molding process would be used for the inventions frame and shelled parts using an appropriate form of plastic and blow molding process would be used for the liquid containing part of the invention using LDPE (low density polyethylene) or MDPE (medium density polyethylene).

This inventions preferred embodiments container is made of a plastic having givable properties but is also configurable with a combination of both givable and rigid plastics constructed in a multiple step molding process with multiple step assembly using such processes as over molding, thermoforming, sonic welding, blow molding and injection molding.

### SUMMARY OF INVENTION

This invention is going to change the way millions of people use eye drops. A new device which constantly warms your liquid eye treatment while continually keeping it protected with anti bacterial radiation. Now there will be no hesitation when you feel the need to use liquid ocular treatments because the discomfort associated with using the liquids is completely eliminated with the use of my invention. When you need to take your eye drops you just reach for the liquid treatment and there it is; warm and ready to drop into your eyes. You can literally give your eyes a warm shower with this invention. A diluted solution will have to be made just for those who wish to use this invention in that way. Currently, there is no known device specifically designed for warming eye drops nor is there a known device which continually keeps your eye drops warm and always ready to use in that warmed state. There is no device available that you can just walk over to at any time, night or day, and pick up a warm and ready container of eye drops from. With my invention, all you need is a power outlet and the invention does the rest. No water is wasted, very little electricity is used, and the benefit to the public in terms of relief is considerable. Warming eye drop bottles in cups, bowls, or whatever someone might use is both unsanitary and just inviting bacteria to infest the bottle which will ultimately contaminate the bottles contents. Eye drop dispensers should really never be heated using water because it just spreads bacteria around like crazy especially water that your fingers have come in contact with. With this invention, the container remains completely dry and bacteria cannot as easily spread or grow on its dry surfaces especially when it's being bombarded by UV/UVB radiation.

A readable microchip, integral to every container produced, will allow new benefits in the fields of ocular treatments that, before this invention, did not exist. A new device which communicates with your bottle of eye drops, protecting you by warning you that your treatments have expired, further protecting your ocular treatments if they contain antibiotics by automatically selecting the proper anti bacterial radiation to give the treatments.

At its heart, this invention is a discomfort relieving/reducing device specifically designed for ocular care. Even though there are many ways to incorporate different electronic components to achieve similar results, the electronics alone are not the invention and should not limit or diminish the essence or the spirit or the scope of this invention and what it accomplishes for the benefit of the public.

Because of this invention, a time consuming, water wasting, energy wasting, unsafe process is no longer necessary for changing the temperature of ocular treatments for the reduction of discomfort when using such treatments. With this new invention, no one has to take extra time out of their day performing a time consuming procedure to make using these treatments bearable. The public now has a better option and a new idea to build on and improve on for years to come.

1) A means for reducing the initial shock and discomfort associated with administering liquid eye treatments whereby; an electronic heating device having a framework formed to receive and be coupled with containers containing liquid ocular treatments receives said containers, heats said containers and said contents preparing said treatments for a more comfortable application to a person's ocular area.

2) The forming of an electronic heater into a shape that accommodates its union with ocular treatment dispensing containers; said electric heater being used for raising the temperature of liquid ocular treatments being stored in said drop dispensers prior to said treatments being expelled from said dispensers in the form of heated liquid drops.

3) A compact electric heater specifically designed for heating the liquid that is contained within liquid drop dispensers.
